(19) United States
(12) Patent Application Publication
(10) Pub. No.: US 2026/0273136 A1
(43) Pub. Date: Sep. 17, 2026

(54) 3D Printed Electronic Skin and Uses Thereof

(71) Applicants: Shounak Roy, College Station, TX (US); Kaivalya Deo, Berkeley, CA (US); Akhilesh K. Gaharwar, Cypress, TX (US)

(72) Inventors: Shounak Roy, College Station, TX (US); Kaivalya Deo, Berkeley, CA (US); Akhilesh K. Gaharwar, Cypress, TX (US)

(73) Assignee: The Texas A&M University System, College Station, TX (US)

(21) Appl. No.: 19/669,826

(22) Filed: May 6, 2026

Related U.S. Application Data
(63) Continuation of application No. PCT/US2024/055206, filed on Nov. 8, 2024.
(60) Provisional application No. 63/597,156, filed on Nov. 8, 2023.

(51) Int. Cl. A61L 27/36; A61L 27/04; A61L 27/20; A61L 27/48; A61L 27/52

(52) U.S. Cl. CPC A61L 27/3637; A61L 27/047; A61L 27/20; A61L 27/48; A61L 27/52; A61L 2430/34

(57) ABSTRACT

Provided herein are hydrogels nanoengineered as a 3D-printed wearable electronic skin conformable to human skin and methods for nanoengineering the hydrogels. The hydrogel may be nanoengineered to have a crosslinked pullulan-SH/polydopamine/molybdenum disulfide network (Pul-SH/PDA/MoS₂). Also provided are methods for detecting and monitoring physiological changes via the wearable electronic skin and for diagnosing a disease or disorder based on the physiological changes.

### CROSS-REFERENCE TO RELATED APPLICATIONS

This continuation application claims benefit of priority under 35 U.S.C. § 120 and 35 U.S.C. § 365 (c) of pending international application PCT/US2024/055206, filed Nov. 8, 2024, which claims benefit of priority under 35 U.S.C. § 119 (e) of provisional application U.S. Ser. No. 63/597,156, filed Nov. 8, 2023, now abandoned, the entireties of both of which is hereby incorporated by reference.

### FEDERAL FUNDING LEGEND

This invention was made with government support under Grant Number W81XWH2210932 awarded by the Peer Reviewed Medical Research Program of the Department of Defense and Grant Number DP2 EB026265 awarded by the National Institutes of Health. The government has certain rights in the invention.

### BACKGROUND OF THE INVENTION

### Field of the Invention

The present invention relates generally to the fields of wearable technology and healthcare. More specifically, the present invention relates to an electronic skin with sensing abilities that mimics the flexibility and stretchability of human skin.

### Description of the Related Art

Flexible bioelectronics have gained attention for their applications in smart electronics, implantable devices, and health monitoring (1-7). Flexible materials are designed to replicate human skin's properties, offering potential applications in robotics, wearable technology, and healthcare. However, the development of such electronic skin, or E-skin, presents numerous challenges including engineering flexible biomaterials, incorporating biosensing capabilities, and utilizing advanced fabrication tools such as 3D printing. Hydrogels present a potential solution for seamless integration due to their tissue-like softness and stretchability, which mirror human tissues in their properties (8-13). By utilizing hydrogel-based electronic skins, interfacial resistance and movement artifacts can be minimized, enabling improved communication between tissue interfaces and devices (14). Traditionally, electronic skins are fabricated by doping conductive polymers or incorporating nanoparticles into crosslinked polymer networks, such as polypyrrole, poly(3,4-ethylenedioxythiophene), graphene, or carbon nanotubes (15-20). However, single-crosslinked hydrogels often lack desirable mechanical properties, resulting in limited stretchability and sensitivity (21).

Thus, there is a need in the art for nanoengineered hydrogel networks suitable as devices for use on human skin. Particularly, the art is deficient in wearable electronic devices integrating biosensors that mimic human skin. The present invention fulfills this longstanding need and desire in the art.

### SUMMARY OF THE INVENTION

The present invention is directed to a hydrogel. The hydrogel comprises a triple crosslinked network of a fungal exopolysaccharide modified with thiol groups, a plurality of 2D transition metal dichalcogenides (TMDs) nanoassemblies having a plurality of sulfur vacancies and a plurality of nanoparticles structured with adhesive properties incorporated into the crosslinked fungal exopolysaccharide-transition metal dichalcogenides.

The present invention also is directed to a method for nanoengineering a triple crosslinked hydrogel network for 3D-printing of an electronic skin. In the method pullulan is modified with thiols to form a thiolated pullulan (Pul-SH). Sulfur vacancies are incorporated in molybdenum disulfide (MoS₂) nanoassemblies to form a defect-rich molybdenum disulfide. The thiolated pullulan is crosslinked with the defect-rich molybdenum disulfide to form a Pul-SH/MoS₂ network. Polydopamine is crosslinked with the thiolated pullulan in the Pul-SH/MoS₂ network to form a Pul-SH/PDA/MoS₂ network. Pullulan chains are crosslinked in the Pul-SH/PDA/MoS₂ network via sodium trimetaphosphate, thereby nanoengineering the triple-crosslinked hydrogel network. The present invention is directed to a related method further comprising printing the triple crosslinked hydrogel network to produce a 3D-printed electronic skin.

The present invention is directed further to a 3D-printed electronic skin produced by the method as described herein.

The present invention is directed further still to a wearable electronic skin. The wearable electronic skin comprises a hydrogel network of thiolated pullulan triple crosslinked via reactions with defect-rich molybdenum disulfide, polydopamine and sodium trimetaphosphate, said crosslinked hydrogel network having a structure of a Pul-SH/PDA/MoS₂ network, and at least one electronic biosensor integrated therein.

The present invention is directed further still to a method for detecting and monitoring physiological changes through skin of a subject. In the method the wearable electronic skin described herein is adhered to the skin of the subject. At least one physiological change in the subject is detected via the at least one electronic biosensor and the at least one physiological change is monitored over a period of time.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1V Show the development of a triple-crosslinked, shear-thinning and tissue-adhesive nanoengineered hydrogels. FIG. 1A is a schematic showing the triple-crosslinking strategy involving defect-driven gelation, Michael addition and ionic crosslinking to develop tissue adhesive, elastomeric and conductive nanoengineered hydrogel. FIGS. 1B-1C are an 1H-NMR analysis of Pul, Pul-DTP and Pul-SH and the quantification of thiol content (%) in Pul-SH derivatives using Ellman's assay. The data are represented as mean±SD (n=3) and statistically examined using a one-way ANOVA test. Statistical significance: *p<0.05, **p<0.01, ***p<0.001 and ns, nonsignificant. FIGS. 1D-1F are Raman spectra of pullulan and Pul-SH. FIGS. 1G-1H are XPS of pullulan and Pul-SH. FIGS. 1I-1J are SEM and TEM images of defect-rich MoS₂ nanoassemblies (1:6). FIG. 1K is a Raman spectra of MoS₂ nanoassemblies.

FIG. 1L-1M show XPS of MoS₂ nanoassemblies. FIG. 1N shows the shear rate sweep of 5 wt % Pul-SH precursor solutions containing various concentrations of MoS₂ nanoassemblies (0.5 wt %, 1 wt %, 2 wt %) showing shear-thinning behavior. FIGS. 1O-1P show the gelation kinetics of 5 wt % Pul-SH solutions in presence of increasing concentrations of MoS₂ nanoassemblies (0.5 wt %, 1 wt %, 2 wt %) and PDA nanoparticles (1 wt %, 2 wt %, 3 wt %). FIG. 1Q shows the shear rate sweep of 5 wt % Pul-SH precursor solutions containing 2 wt % each of MoS₂ nanoassemblies and PDA nanoparticles showing shear-thinning behaviour. FIG. 1R shows gelation kinetics of 5 wt % Pul-SH solutions in absence and presence of MoS₂ nanoassemblies and PDA nanoparticles confirming defect-driven gelation and Michael addition between the polymer and the crosslinkers. FIGS. 1S-1V show the oscillatory stress sweep of single, dual and triple crosslinked hydrogels containing various concentrations of MoS₂ nanoassemblies (0.5 wt %, 1 wt %, 2 wt %) showing mechanical reinforcement with each crosslinking step.

FIGS. 2A-2K show the physico-chemical characterization of crosslinked Pul-SH/PDA/MoS₂ hydrogel. FIG. 2A is a microstructure characterization of triple-crosslinked nanoengineered hydrogels. SEM images of transverse sections of freeze-dried Pul-SH/PDA, Pul-SH/MoS₂, Pul-SH/PDA/MoS₂ and triple crosslinked hydrogels showing porous morphology of the hydrogels with interconnected networks. FIGS. 2B-2D are EDX mapping and EDS spectra of the hydrogel showing uniform distribution of Mo and S atoms in the hydrogel. FIGS. 2E-2J are deconvoluted C1s and Mo3d XPS spectra of Pul-SH, Pul-SH/PDA, Pul-SH/MoS₂ and Pul-SH/PDA/MoS₂ showing successful crosslinking reactions via defect-driven gelation and Michael addition. The C1s spectra of crosslinked hydrogels show a decrease in the intensity of C—S peak at 286.7 eV in comparison to un-crosslinked Pul-SH due to the formation C—S—Mo and C—S-PDA. Similarly, in the Mo3d spectra appearance of a new peak at 235.2 eV can be seen due to the formation of MoS₂-Pul-SH crosslinks. FIG. 2K are Raman spectra of Pul-SH, Pul-SH/PDA, Pul-SH/MoS₂ and Pul-SH/PDA/MoS₂ further demonstrating chemical crosslinking. Peak intensities corresponding to C—S stretching, thiol bending and stretching significantly decreased in crosslinked hydrogels.

FIGS. 3A-3P show the 3D printability of nanoengineered ink and mechanical characterization of triple-crosslinked nanoengineered hydrogel. FIGS. 3A-3B show stretching and elongation tensile testing of nanoengineered hydrogels at different MoS₂ concentrations and their Young's modulus. The data are represented as mean±SD (n=3) and statistically examined using a one-way ANOVA test. Statistical significance: *p<0.05, **p<0.01, ***p<0.001, ****p<0.0001 and ns, nonsignificant. FIGS. 3C-3D show cyclic compression testing of Pul-SH/2 wt % PDA/1 wt % MoS₂ hydrogel at 20%, 40%, 60%, and 80% strain showing successful compression and recovery of hydrogels at each cycle. FIGS. 3E-3G show different crosslinking chemistries (single, dual and triple crosslinking) and increasing MoS₂ concentrations (0.5%, 1% and 2%) increase the compressive modulus and toughness of hydrogels demonstrating mechanical reinforcement and robustness. FIG. 3H shows the maximum stress values of the nanoengineered hydrogel FIGS. 31-3P show 3D extrusion printability of nanoengineered hydrogel ink. The printed structures of curved cylindrical lines, circular loop, crosshatch pattern, hollow and solid star, TAMU logo, and hollow cylinder demonstrates versatility of the nanoengineered ink towards 3D printing. The data are represented as mean±SD (n=3) and statistically examined using a one-way ANOVA test. Statistical significance: *p<0.05, **p<0.01, ***p<0.001, *p<0.0001 and ns, nonsignificant.

FIGS. 4A-4L show the electrical conductivity of nanoengineered hydrogel and its application as an electronic skin for dynamic sensing of strain and pressure. FIG. 4A shows that electrical impedance spectroscopy (EIS) was performed on the Pul-SH/PDA/MoS₂ nanoengineered hydrogels and impedance quantification reveals optimum inclusion of MoS₂ nanoassemblies leads to decrease in electrical resistance and increase in conductivity. The electrical conductivity of 3D printed hydrogel precursors patterns and crosslinked hydrogels was demonstrated through a closed electrical circuit illuminating LEDs. FIGS. 4B-4C show the relative change in resistance values (ΔR/R₀) of Pul-SH/PDA/MoS₂ hydrogel in response to multiple cycles of compression (25 cycles) and changes in external force/strain/pressure, demonstrating linearity in response. FIGS. 4D-4G show the relative change in resistance values (ΔR/R₀) of 3D-printed Pul-SH/PDA/MoS₂ wearable device in response to bending at 45° and 30°, double bending at 90° in both upward and downward direction, rapid single-side bending at 45° and twisting motion, over multiple bending cycles. FIGS. 4H-4K The elastomeric hydrogels were able to precisely respond to dynamic gentle and hard press. Blowing of air on the hydrogel surface can also be detected through a change in resistance for each blow cycle. The electronic skin was attached to the throat and vocal cord vibrations of two distinct activates of coughing and drinking water was precisely recorded. FIG. 4L shows that the 3D printed hydrogels may also be utilized as flexible touch keyboard wherein each alphabet of the word TAMU could be uniquely identified.

FIGS. 5A-5E show the 3D printed electronic skin as a flexible temperature sensor. FIG. 5A shows the relative change in resistance values (ΔR/R₀) of 3D-printed Pul-SH/PDA/MoS₂ electronic skin as a function of temperature (0° C.-60° C.). FIG. 5B shows the reproducible temperature-discrimination capacity of the electronic skin during repetitive cold (22° C.) and heat (50° C.) source approaching. FIG. 5C shows the transient thermosensitive ability of the electronic skin to hot (50° C.) and cold (8° C.) water. FIG. 5D shows the ability of the electronic skin to measure human fever range temperature (98° F.-104° F.). FIG. 5E shows the real-time monitoring of body temperature using electronic skin as a flexible thermometer.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant FIG.

In one embodiment of the present invention, there is provided a hydrogel, comprising a triple crosslinked network of a fungal exopolysaccharide modified with thiol groups; a plurality of 2D transition metal dichalcogenides (TMDs) nanoassemblies having a plurality of sulfur vacancies; and a plurality of nanoparticles structured with adhesive properties incorporated into the crosslinked fungal exopolysaccharide-transition metal dichalcogenides.

In this embodiment, the modified fungal exopolysaccharide may be a thiolated pullulan (Pul-SH). Also, in this embodiment the 2D transition metal dichalcogenides nanoassemblies may comprise molybdenum disulfide (MoS₂). In addition, the plurality of nanoparticles may be polydopamine (PDA) nanoparticles. Particularly, the hydrogel may comprise a crosslinked pullulan-SH/polydopamine/molybdenum disulfide network (Pul-SH/PDA/MoS₂).

In another embodiment of the present invention, there is provided a method for nanoengineering a triple crosslinked hydrogel network for 3D-printing of an electronic skin, comprising modifying pullulan with thiols to form a thiolated pullulan (Pul-SH); incorporating sulfur vacancies in molybdenum disulfide (MoS₂) nanoassemblies to form a defect-rich molybdenum disulfide; crosslinking the thiolated pullulan with the defect-rich molybdenum disulfide to form a Pul-SH/MoS₂ network; crosslinking polydopamine with the thiolated pullulan in the Pul-SH/MoS₂ network to form a Pul-SH/PDA/MoS₂ network; and crosslinking pullulan chains in the Pul-SH/PDA/MoS₂ network via sodium trimetaphosphate, thereby nanoengineering the triple-crosslinked hydrogel network. Further to this embodiment, the method comprises printing the triple crosslinked hydrogel network to produce a 3D-printed electronic skin.

In yet another embodiment of the present invention, there is provided a 3D-printed electronic skin produced by the method as described supra.

In this embodiment, the 3D-printed electronic skin may comprise an integrated dynamic strain, pressure, and temperature sensor. Also, in this embodiment the 3D-printed electronic skin may be a flexible strain sensor configured to detect different dynamic human motions. In addition, the 3D-printed electronic skin may be a wearable phonatory recognition platform and flexible touch keypad configured to detect precise changes in pressure. Furthermore, the 3D-printed electronic skin may be a flexible thermometer configured to detect precise changes in temperature. Further still, the 3D-printed electronic skin has the properties of flexibility, stretchability, adhesion, moldability, and electrical conductivity when adhered to human skin.

In yet another embodiment of the present invention, there is provided a wearable electronic skin comprising a hydrogel network of thiolated pullulan triple crosslinked via reactions with defect-rich molybdenum disulfide, polydopamine and sodium trimetaphosphate, where the crosslinked hydrogel network has a structure of a Pul-SH/PDA/MoS₂ network, and at least one electronic biosensor integrated therein.

In this embodiment, the electronic biosensor may be configured as a motion tracker, thermometer, phonatory-recognition platform, or touchpad or a combination thereof. Also, in this embodiment the Pul-SH/PDA/MoS₂ network may be conformable to human skin.

In yet another embodiment of the present invention, there is provided a method for detecting and monitoring physiological changes through skin of a subject, comprising adhering the wearable electronic skin of claim 14 to the skin of the subject; detecting at least one physiological change in the subject via the at least one electronic biosensor; and monitoring the at least one physiological change over a period of time.

Further to this embodiment, the method comprises recording the physiological change. Further still to this embodiment, the method comprises diagnosing a disease or disorder based on the at least one physiological change in the subject. In all embodiments, the at least one electronic biosensor may detect human motions, senses touch, phonatory vibrations and body temperature.

Provided herein are nanoengineered hydrogel networks and 3D-printed electronic skin made from the hydrogel networks. Particularly, the 3D printed electronic skin is fabricated utilizing triple crosslinked nanoengineered hydrogels with tunable mechanical properties with electronic and thermal sensing capabilities via at least one biosensor integrated therein. The engineered e-skin is flexible, moldable, stretchable, adhesive with utility in electronic wearable skin, soft robotics and actuators. Also provided are methods for nanoengineering the hydrogel networks.

The hydrogel networks are networks of a modified fungal exopolysaccharide, such as, but not limited to, thiolated pullulan, that is produced via a triple crosslinking with crosslinking agents to engineer a hydrogel network that simulates properties of human skin. Non-limiting examples of these crosslinking agents are nanoassemblies of or nanomaterials comprising defect rich 2D transition metal dichalcogenides, for example, molybdenum disulfide (MoS₂) nanoassemblies with a plurality of sulfur vacancies, polydopamine and sodium trimetaphosphate.

The ability of the hydrogel to adhere to human skin enables monitoring and/or measuring applications when placed on the skin. The electronic skins provided herein are useful as biosensors integrated to sense or precisely detect and measure dynamic strain, pressure, and temperature. In non-limiting examples the electronic skin may be integrated with the hydrogel network as a flexible strain sensor that is configured to detect different dynamic human motions, as a wearable phonatory recognition platform and flexible touch keypad configured to detect precise changes in pressure or as a flexible thermometer configured to detect precise changes in temperature. Thus, provided are methods for detecting, monitoring and recording at least one physiological change in the subject via the biosensors in the electronic skin and methods for diagnosing a disease or disorder in the subject based on or associated with the physiological change(s).

The following examples are given for the purpose of illustrating various embodiments of the invention and are not meant to limit the present invention in any fashion.

### Example 1

### Methods and Materials

### Synthesis of Thiolated Pullulan (Pul-SH):

Thiolated pullulan was synthesized through the process of esterification of pullulan using 3,3′-dithiopropionic acid (DTPA), followed by reduction with dithiothreitol (DTT) to obtain free thiol. 0.75 g of Pullulan, 1.2 g DTPA and 0.35 g 4-(Dimethylamino)pyridine (DMAP) was added into a round bottom flask and purged with nitrogen. To it, 37.5 mL of anhydrous DMSO was added and the reagents were thoroughly mixed. Once all the reagents have fully dissolved, a predetermined volume of Boc₂O was added drop-wise to the solution under magnetic stirring. Finally, the reaction was allowed to take place at 45° C. for 24 h under nitrogen atmosphere. The mixture was then dialyzed against distilled water for 3 days and lyophilized to get a solid product, Pul-DTP. Next, 0.5 g of Pul-DTP was dissolved in 50 mL of distilled water and to it 0.5 g of DTT was added. The pH of the solution was set to 8.5 with 0.1 N NaOH and the reaction was allowed to take place for 3 h at room temperature under continuous stirring. To stop the reaction, the pH of the solution was brought to 3.5 using 0.1 N HCl. This solution was first dialyzed for 2 days against acid-water and then for 1 day in normal distilled water to remove the unreacted molecules. Finally, the dialyzed product was lyophilized to obtain thiolated pullulan, Pul-SH.

To synthesize different derivatives of Pul-SH having varying thiol content, the amount of Boc₂O was varied in the reaction mixture to get Pul-SH-1 (Boc₂O=100 μL), Pul-SH-2 (Boc₂O=200 μL) and Pul-SH-3 (Boc₂O=350 μL). The degree of thiolation in Pul-SH was determined using Ellman's assay. 0.1 M sodium phosphate buffer (pH=8.0) containing 1 mM EDTA was used as the reaction buffer. Pul-SH and control pullulan was dissolved in reaction buffer at a concentration of 10 mg/mL. Subsequently, 500 μL of sample, 100 μL of Ellman's reagent (4 mg/mL) and 5 mL of reaction buffer were mixed and incubated at 37° C. for 20 min for the color to develop. The absorbance of the samples was taken at 412 nm and the thiol grafting % was calculated from a standard curve of DTT.

### Synthesis of Defect-Rich MoS₂ Nanoassemblies

The 1:6 defect-rich MoS₂ nanoassemblies were synthesized through a hydrothermal process according to the method previously published by Jaiswal et al. Briefly, 1 mmol of hexaammonium heptamolybdate tetrahydrate, (NH₄)₆·Mo₇O₂₄·4H₂O, and 42 mmol of thiourea, NH₂CSNH₂ were dissolved in 50 mL of DI water, vortexed thoroughly and transferred to a teflon-lined hydrothermal vessel. The reaction was carried out at 220° C. for 18 h, cooled to room temperature and then washed several times with ethanol and water to remove impurities. Finally, the solid product was vacuum dried overnight to obtain powders of MoS₂ nanoassemblies.

### Synthesis of PDA Nanoparticles

In a beaker, 45 mL of ethanol and 4.5 mL of ammonium hydroxide (NH₄OH) were added to 95 ml distilled water and stirred for 30 min at room temperature. To this solution, a 10 ml solution of dopamine hydrochloride (50 mg/mL) was added and stirred for 12 h at room temperature. Subsequently, the solution was washed several times with ethanol and water to remove impurities and the solid product was vacuum dried to obtain powders of PDA.

### Synthesis of Triple Crosslinked Nanoengineered Hydrogel (Pul-SH/PDA/MoS₂)

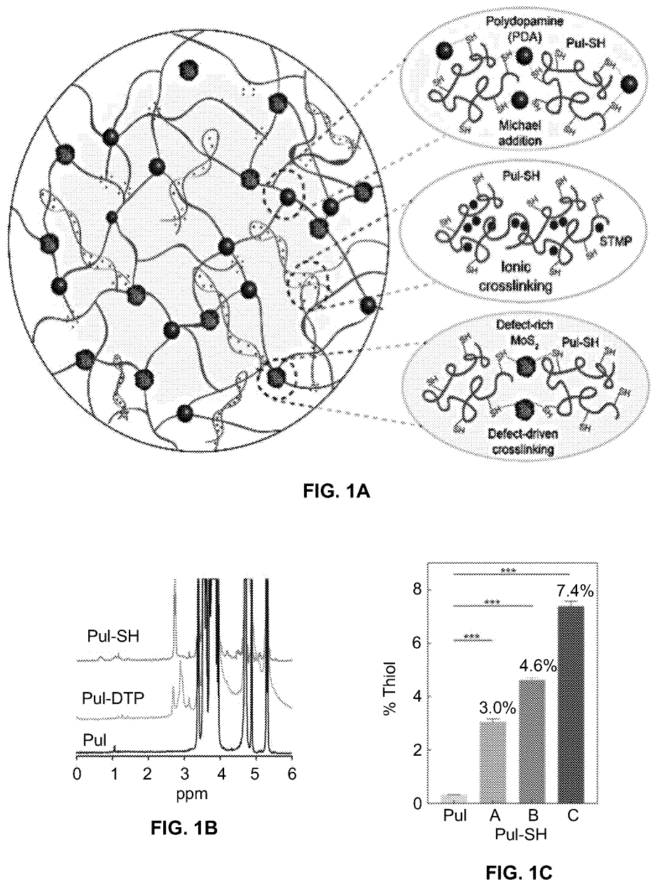

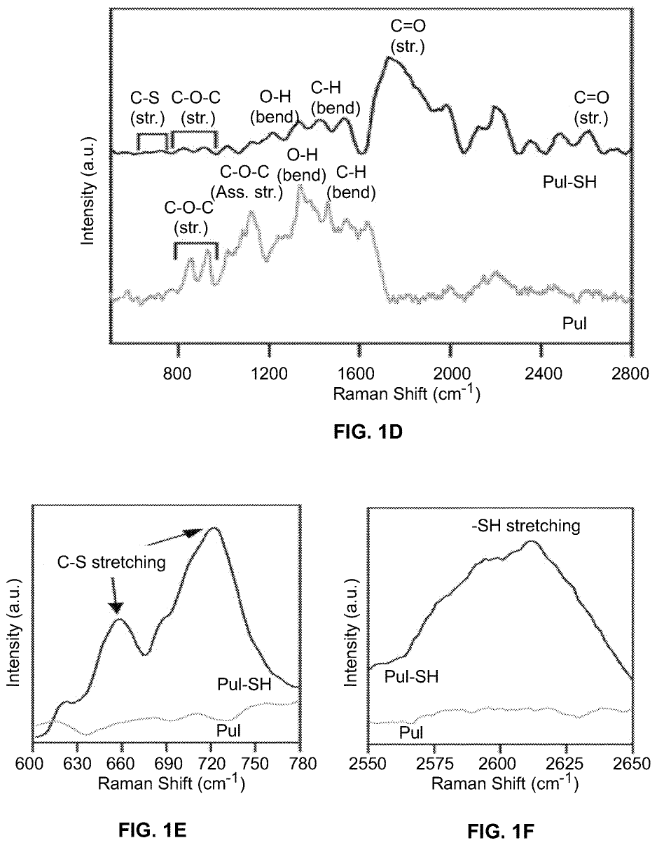

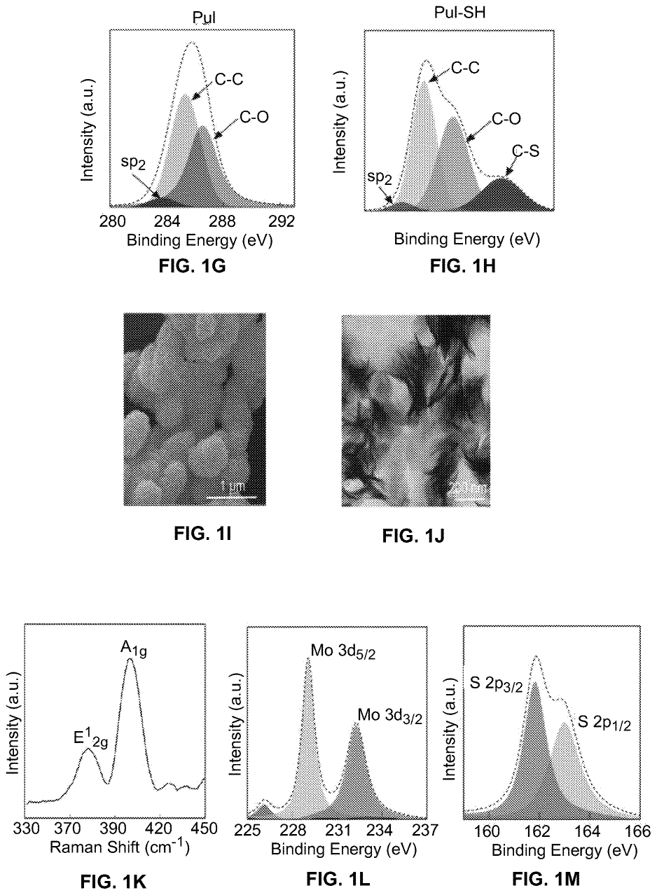

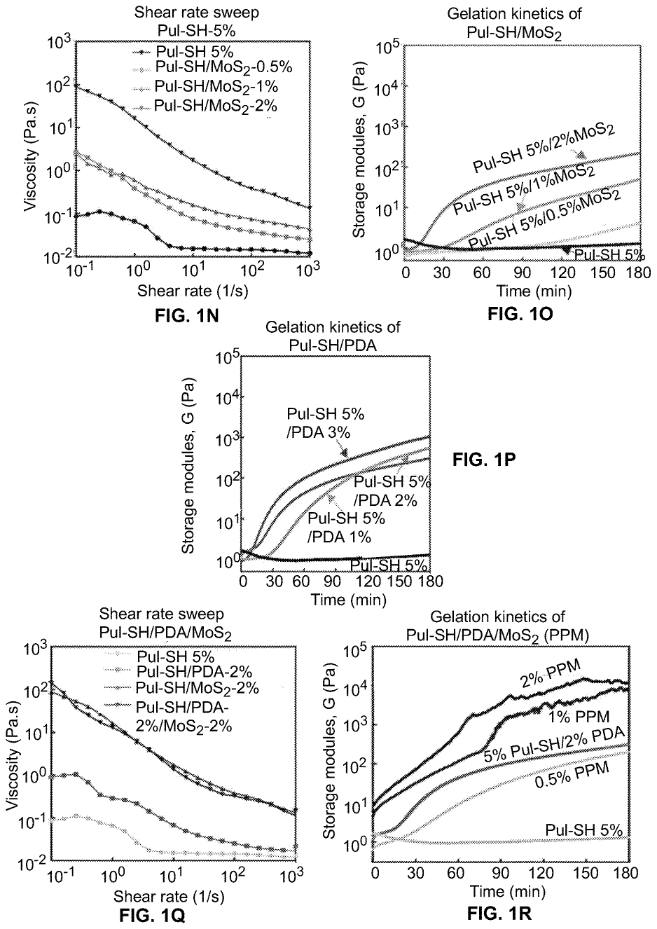

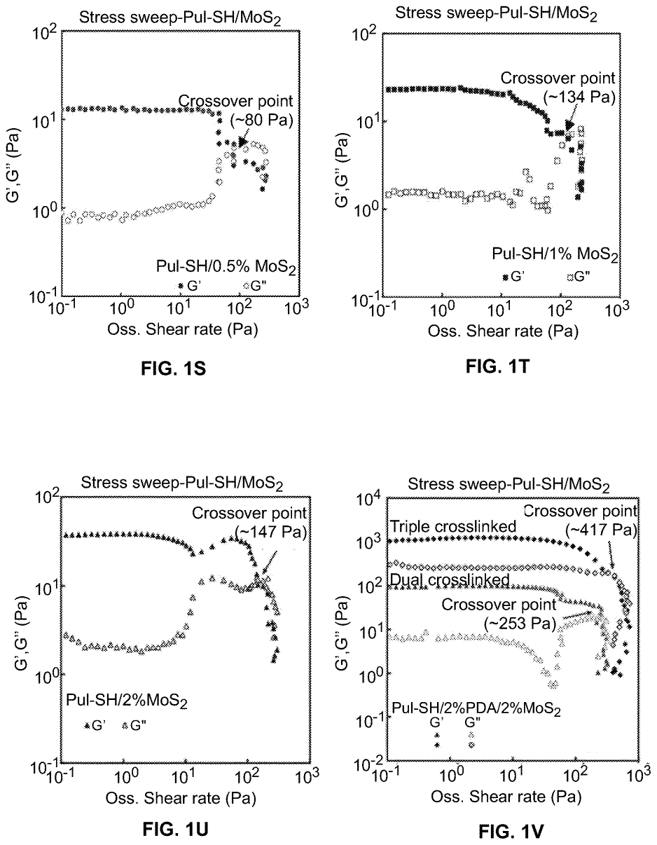

The triple crosslinked nanoengineered hydrogel was synthesized through covalent crosslinking of Pul-SH with defect-rich MoS₂ nanoassemblies and PDA nanoparticles, and ionic crosslinking using sodium trimetaphosphate (STMP). 5 wt % Pul-SH solution was mixed with MoS₂ nanoassemblies (0.5-2 wt %), PDA nanoparticles (1-3 wt %) and STMP (5% v/v) in a glass vial and vortexed for 1 min to uniformly mix the different components (FIG. 1A). This hydrogel precursor solution was then allowed to gel at 37° C. for 1 h to obtain the crosslinked hydrogels. The precursor solution was also injected into molds to get hydrogels of different shapes and sizes. The obtained crosslinked hydrogels were soaked in PBS for removing any unreacted components.

### Characterization of Hydrogel Precursors and Crosslinked Hydrogel

Successful thiolation of Pul-SH was characterized using 1H-NMR (AVANCE NEO 400), Raman spectroscopy (LabRam HR confocal Raman microscope, Horiba Inc., Japan) and XPS (Omicron XPS system with an Argus detector) analysis. The thiol % of Pul-SH was determined using Ellman's assay. The morphology of the defect-rich MoS₂ nanoassemblies were characterized using SEM (FEI Quanta 600 FE-SEM) and TEM (JEOL 2010, 200 keV). The chemical composition of the MoS₂ nanoassemblies was investigated through Raman spectroscopy (LabRam HR confocal Raman microscope, Horiba Inc., Japan) and XPS (Omicron XPS system with an Argus detector) analysis. The microstructure and porous morphology of crosslinked hydrogels was studied using SEM (FEI Quanta 600 FE-SEM). The hydrogels were freeze dried and thin transverse sections of the hydrogels were prepared for imaging. The sections were plasma coated with a 10 nm thick Pt/Pd coating to improve conductivity of the samples and imaged under SEM. The elemental composition and distribution of Mo and S atoms in the hydrogel was carried out using EDS mapping. The mechanism of gelation between Pul-SH, MoS₂ nanoassemblies and PDA nanoparticles was probed through Raman spectroscopy (LabRam HR confocal Raman microscope, Horiba Inc., Japan) and XPS (Omicron XPS system with an Argus detector) analysis. The binding energies for carbon (1s), sulfur (2p), and molybdenum (3d) were recorded and further deconvoluted and processed by CasaXPS multiple peak fit software.

### Rheology

The shear-thinning behavior, gelation kinetics and oscillatory-stress sweep characterization were carried out at room temperature using a stress-controlled rheometer (DHR-2 Discovery hybrid rheometer, TA Instruments, New Castle, DE, USA) having a 20 mm parallel plate geometry and 0.2 mm gap. Shear-thinning and gelation kinetics were carried out on hydrogel prepolymer solutions, whereas oscillatory-stress sweep characterization was carried out on cylindrical shaped crosslinked hydrogels having a dimension of 5 mm×5 mm.

### Compression and Tensile Testing

The compressive modulus of cylindrical shaped crosslinked hydrogels having a dimension of 5 mm×5 mm was determined using a cyclic compression testing in a ADMET eXpert 7600 system (ADMET, Inc., Norwood, MA, USA) with an attached load cell of 25 lb. Variable strains ranging from 20% to 80% was applied on the hydrogels at a strain rate of 1 mm/min and the compressive modulus was calculated corresponding to the 10-20% strain region of the stress-strain curve (dividing force by the original cross-section area). For tensile/elongation testing, rectangular hydrogels with dimensions of 15×5×1 mm (length×width×thickness) were fabricated and tensile testing at a rate of 1 mm/min was performed on these hydrogels using an Instron 5944 Universal Testing System equipped with tensile clamps. From the resulting stress-strain curves, The Young's modulus (E) of the hydrogels was calculated from the stress-strain curves.

### Adhesiveness Test

The adhesive property of the crosslinked hydrogels was evaluated against a wide variety of materials such as metal, plastic, glass, rubber and human skin. Hydrogels were placed on different surfaces and their adhesiveness was evaluated based on the ability of the hydrogels to remain attached to the surfaces even after inverting them upside down.

### 3D Printing

Different 2D and 3D shapes were 3D printed by an extrusion-based modified Anet A8 printer. Shapes were designed in Solidworks and then fed into Slic3r as STL files. The printing variables were customized and converted into G-code instructions for the printer. The printing was done at a speed of 5 mm/s with a 410 μm tapered tip attached to the extruder. The layer height during printing was kept at 200 μm and the layer width was set at 300 μm. A number of different shapes such as curved lines, a circular band, a square crosshatch grid, a hollow and solid five-pointed star, university logo and a solid cylinder with a height of 10 mm were printed.

### Electrical Signal Measurement and Resistance-Change Monitoring

The electrical properties of the hydrogels at varying concentration of MoS₂ nanoassemblies (0, 0.5, 1 and 2 wt %) was measured via two-electrode EIS. Hydrogels disks (5 mm×5 mm) were hydrated in 0.1M PBS (pH=7.4) at RT, and their conductivity was measured by sandwiching them between a pair of circular (1)=8 mm), gold-coated, polyamide electrodes that were cleaned in methanol. EIS measurements were done at an interrogation voltage of 20 mV p-to-p and over the frequency range of 0.01 Hz to 10 MHz at RT. The response of the hydrogels towards different dynamic motions of strain (30°, 45° and 90° bending, twisting) and pressure (gentle/hard press, blowing of air, coughing, swallowing of water and writing) were measured in terms of real-time resistance change using a Mark-10 force gauge series 5 and ESM 303 (Mark-10 Corp., Copiague, NY, USA) and NI USB-4065 digital multimeter (NI Corp., Austin, TX, USA).

### Thermosensitive Performance Measurement

The response of the hydrogels towards hot and cold temperature was also measured in terms of real-time resistance change using a Mark-10 force gauge series 5 and ESM 303 (Mark-10 Corp., Copiague, NY, USA) and NI USB-4065 digital multimeter (NI Corp., Austin, TX, USA). Hydrogels were brought in direct contact with hot and cold sources maintained at a fixed temperature and the change in conductivity of the hydrogels in response to each temperature was recorded in terms of relative resistance change. An infrared camera (FLIR, Arlington, VA) was used for measuring the human body temperature.

### Statistical Analysis

All data are represented as the mean±standard deviation (SD) of triplicate samples (n=3-10). Comparative analysis between groups was performed by a one-way analysis of variance (ANOVA) test. *p<0.05, **p<0.01, *p<0.001, *p<0.0001 and ns, nonsignificant.

### Human Subject

The potential risks of the study were explained to recruited human subjects, and informed consent, including publication of human subject photographs, was obtained after the procedure.

### Example 2

### Results

### Synthesis and Characterization of Injectable and Adhesive Nanocomposite Hydrogels

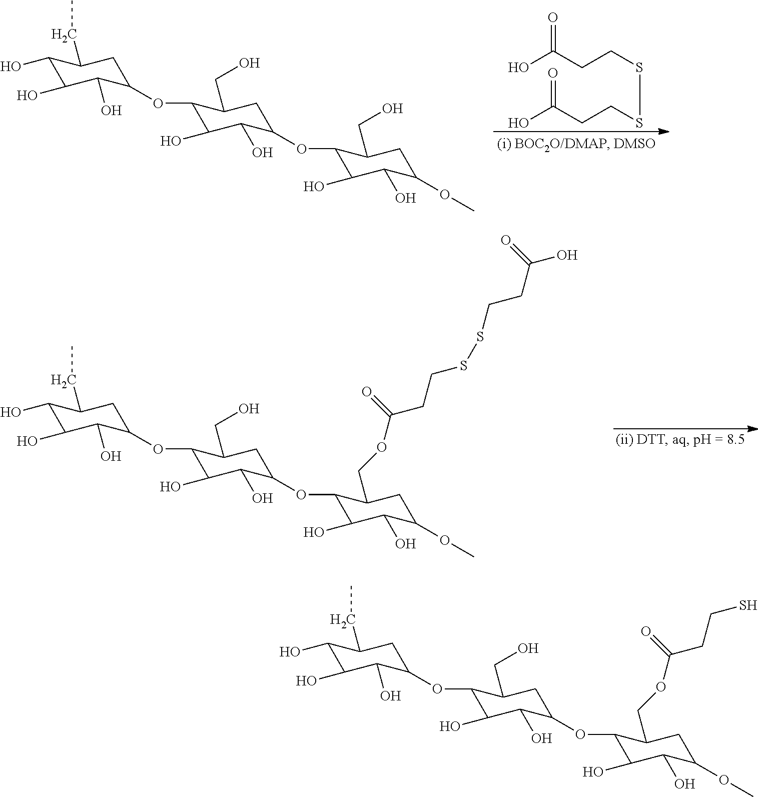

Thiolated-pullulan (Pul-SH) was synthesized via a two-step process involving esterification followed by DTT reduction (26). Briefly, pullulan was first reacted with 3,3′-dithiopropionic acid (DTPA) to yield an intermediate macromer, Pul-DTP, which formed as a result of coupling of the DTP moieties to the hydroxyl groups of pullulan. The disulfide group present in Pul-DTP was subjected to DTT reduction in order to obtain free thiol groups on pullulan (Pul-SH). 1H-NMR analysis of the intermediate macromer, Pul-DTP and the final product Pul-SH revealed the functionalization of pullulan with thiol groups (FIGS. 1B-1C). The presence of ethyl multiplet peaks (o=2.62-2.91) in the spectrum of Pul-DTP indicated successful incorporation of DTP moieties. After DTT reduction, Pul-SH showed a singlet peak at 0=2.7-2.8, confirming the presence of thiol groups (FIGS. 1B-1C) (26). The absence of ethyl multiplet and singlet peaks in the spectrum of unmodified pullulan further confirmed thiolation of pullulan (Pul-SH).

Raman (FIGS. 1D-1F) and X-Ray photoelectron spectroscopy (XPS) (FIGS. 1G-1H) analysis were performed to further validate the thiolation process of Pul-SH. Raman spectra of pullulan exhibited characteristic peaks at 1471 cm⁻¹ and 1352 cm⁻¹ corresponding to C—H bending and O—H bending vibrations, respectively. Peaks associated with asymmetric C—O—C(1132 cm⁻¹ and 941 cm⁻¹) and symmetric C—O—C stretching (858 cm⁻¹) vibrations were observed (27). The Raman spectrum of Pul-SH displayed additional peaks at 660 cm⁻¹, 720 cm⁻¹ and 2610 cm⁻¹, which correspond to C—S stretching and —SH stretching vibrations respectively, indicating the presence of thiol groups (22, 28). A peak at 1752 cm-1, which corresponding to C═O stretching from the DTP moiety structure was also observed. XPS analysis of pullulan revealed peaks at 283 eV, 284.7 eV, and 285.6 eV, representing sp², C—C, and C—O groups, respectively. In the case of Pul-SH, these peaks shifted, and a new peak at 286.7 eV corresponding to the C—S group emerged, confirming the thiolation of pullulan (FIGS. 1G-1H) (28). By varying the concentration of DTPA, a series of Pul-SH derivatives with different thiol content was synthesized. Thiol content in these derivatives was determined to range from 3-7.4% using Ellman's assay. (FIGS. 1B-1C). The derivative Pul-SH-C with a thiol content of 7.4% was chosen for hydrogel preparation.

MoS₂ nanoassemblies with high atomic defect density, specifically sulfur vacancies, were synthesized using our hydrothermal approach (22). SEM and TEM analysis revealed a flower-like morphology with an average size of ~200-500 nm, where each petal represents a MoS₂ nanosheet (FIGS. 1I-1J). This unique structure prevents restacking of the nanosheets, providing easy access to the atomic defects and a large surface area. Raman analysis showed characteristic bands at 376.5 cm-1 (E¹_{2g}) and 406 cm⁻¹ (A_{1g}) corresponding to the in-plane and out-of-plane vibrations of sulfur atoms, respectively (FIG. 1K). XPS analysis confirmed the chemical composition with binding energies at 232.73 eV (Mo 3d_{3/2}), 229.63 eV (Mo 3d_{5/2}), 161.16 eV (S 2p_{3/2}), and 163.25 eV (S 2p_{1/2}) for the respective elements (FIGS. 1L-1M). These results validate the chemical nature of the synthesized MoS₂ nanoassemblies.

The incorporation of defect-rich MoS₂ nanoassemblies and PDA nanoparticles into Pul-SH solution resulted in formation of a crosslinked network. The interaction of prepolymer solution (5 wt % of Pul-SH) with MoS₂ nanoassemblies and PDA nanoparticles was studied by measuring the viscosity of the prepolymer solutions at different shear rates. MoS₂ nanoassemblies interacted strongly with the prepolymer solution and increased the viscoelastic characteristics to Pul-SH in a concentration dependent manner (FIGS. 1N-1P), which was otherwise absent in Pul-SH. A quantitative estimation of the observed viscoelastic property was carried out by fitting power law model to understand the shear-thinning characteristics of the prepolymer solution (Table 1) (22).

TABLE 1
Power law index

| | Consistency | |
|---|---|---|
| | coefficient (K) | Power law index (n) |
| Pul-SH | 0.0516 | 0.72 |
| Pul-SH/MoS2-0.5 | 0.4466 | 0.486 |
| Pul-SH/MoS2-1 | 0.6158 | 0.553 |
| Pul-SH/MoS2-2 | 10.15 | 0.351 |
| Pul-SH/PDA-2 | 0.2638 | 0.538 |
| Pul-SH/PDA/MoS2-2 | 12.625 | 0.244 |

The power law index (n) serves as a parameter to determine shear-thinning behavior. For Newtonian fluids, n=1, however for fluids with shear-thinning properties, the value of n ranges between 0 to 1 (22). At a low shear rate of 0.1 s⁻¹, the viscosity of Pul-SH/2% MoS₂ was 102 Pa·s, which decreased linearly to about 10⁻¹ Pa·s at a higher shear rate of 1000 s⁻¹ (n=0.35). Similar shear-thinning property were observed for other MoS₂ concentrations of 0.5 wt % and 1 wt %, which yielded a n value of 0.48 and 0.55, respectively (Table 1). However, Pul-SH (5 wt %) in the absence of MoS₂ nanoassemblies did not show a shear-thinning behavior between the shear rates of 0.1-1000 s⁻¹. PDA alone did not play a role in imparting shear-thinning to Pul-SH (FIGS. 1Q-1R), but contributed towards increasing the viscosity of the prepolymer solution. In the presence of both MoS₂ and PDA, Pul-SH/2% PDA/2% MoS₂ showed significant shear-thinning behaviour with a n value of 0.24 (FIG. 1Q-1R).

The process of hydrogel formation was studied by monitoring the gelation kinetics of Pul-SH over a period of time (FIG. 1Q-1R). Increase in storage modulus of Pul-SH over time in the presence of MoS₂ nanoassemblies and PDA nanoparticles indicate the formation of crosslinked networks. For example, the storage modulus of Pul-SH/2% MoS₂ increased from 1 Pa to 50 Pa within 60 min after which it attained a plateau (FIGS. 1N-1P). Similarly, interaction of 3 wt % PDA with Pul-SH also resulted in an increase in the storage modulus from 1 Pa to 100 Pa within 60 min followed by a plateau (FIGS. 1N-1P). Furthermore, the addition of both MoS₂ nanoassemblies and PDA together into Pul-SH resulted in faster formation of crosslinked viscoelastic networks (Pul-SH/PDA/MoS₂; PPM) within ~30-45 min (FIG. 1Q-1R), where the free thiol groups of Pul reacted with both the sulfur vacancies of MoS₂ and catechol groups of PDA to form the hydrogel faster. The digital photographs in the inset clearly shows the gelation phenomenon. A 5 wt % solution of Pul-SH in the absence of MoS₂ nanoassemblies and PDA nanoparticles did not show any gelation.

Next, the network stability of the crosslinked hydrogel along with the individual effect of three crosslinking chemistries on hydrogel stability and mechanical integrity was investigated using an oscillatory stress-sweep experiment (FIGS. 1S-1V). A Pul-SH hydrogel made with 0.5 wt % MoS₂ nanoassemblies showed a yield stress of 80 Pa (FIGS. 1S-1V), which increased to 134 Pa in presence of 1 wt % MoS₂ nanoassemblies (FIGS. 1S-1V) and further increased to 147 Pa with 2 wt % MoS₂ nanoassemblies (FIGS. 1S-1V), thereby showing the ability of MoS₂ nanoassemblies to impart network stability to crosslinked hydrogel. Moreover, dual-crosslinking of Pul-SH hydrogel by both 2 wt % MoS₂ nanoassemblies and 2 wt % PDA resulted in a much higher yield stress of ~253 Pa, thereby further improving the network stability (FIGS. 1S-1V). Finally, in the presence of all three crosslinking chemistries, namely defect-driven gelation, Michael addition and ionic crosslinking, the resultant hydrogel showed high mechanically stability that can withstand a shear stress of ~417 Pa without undergoing deformation (FIGS. 1S-1V). These results thus establish that triple crosslinking work together to significantly reinforce the nanoengineered hydrogel network. The incorporation of PDA into the hydrogel is expected to impart adhesiveness to the hydrogel surface that can be utilized for designing a flexible bioelectronic device. The triple-crosslinked pullulan hydrogel demonstrated excellent adhesiveness to a wide range of substrates such as metal, plastic, rubber, glass and even human skin, thereby showing the suitability of using this hydrogel system as a tissue-adhesive electronic skin.

### Defect-Driven Gelation and Michael Addition Mediated Crosslinking

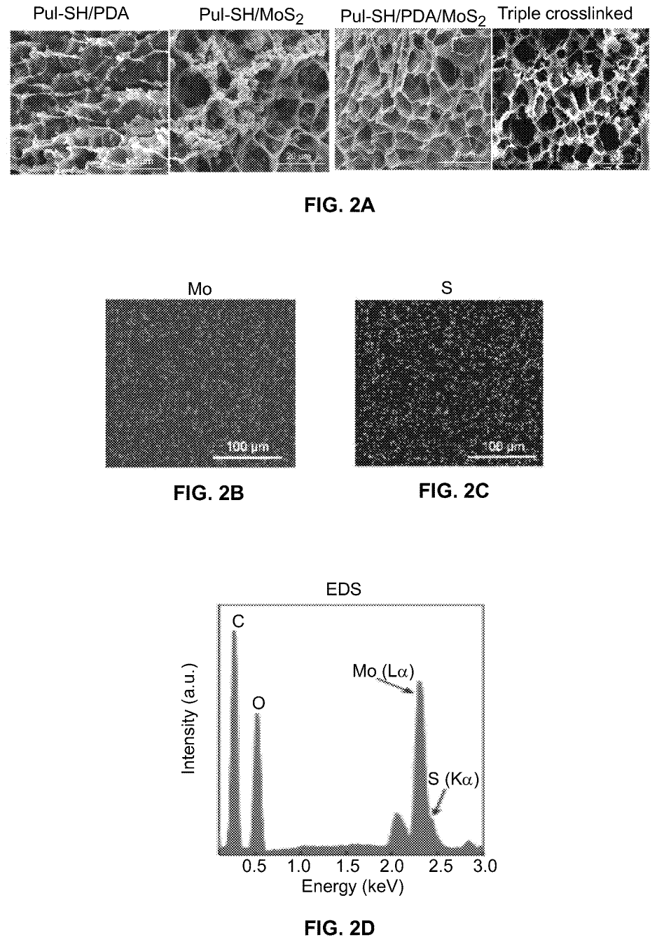

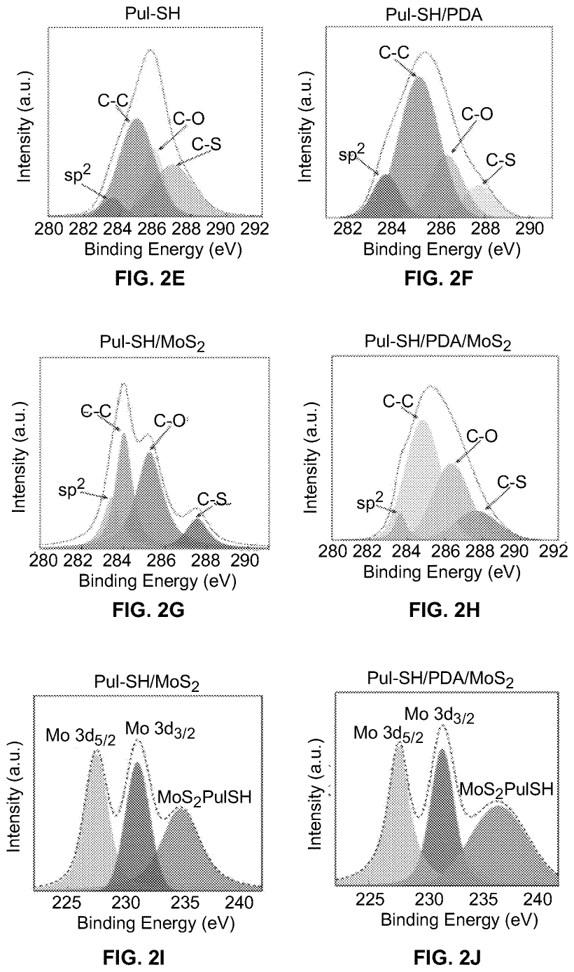

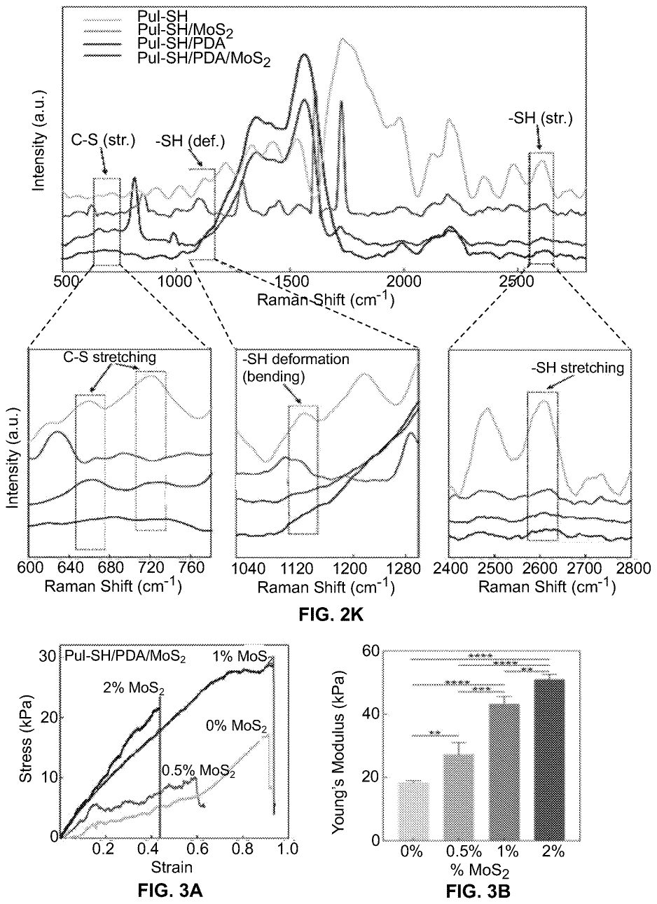

The microstructure of the freeze-dried hydrogels was studied using SEM. The SEM images show the successful integration of pullulan with MoS₂ nanoassemblies, and PDA nanoparticles to form an interconnected porous network (FIG. 2A). We further carried out energy-dispersive X-ray spectra (EDS) mapping of the hydrogel sections to confirm the presence of MoS₂ nanoassemblies. The EDS mapping (FIGS. 2B-2D) results clearly showed the uniform distribution of Mo and S atoms in the hydrogel network.

To confirm the formation of covalent bonds between Pul-SH, MoS₂ nanoassemblies and PDA nanoparticles, the chemical composition of crosslinked hydrogels was investigated using XPS (FIGS. 2E-2J). The deconvoluted C1s XPS spectra of un-crosslinked Pul-SH showed peaks at 283, 284.7 and 285.6 eV corresponding to sp², C—C and C—O groups in pullulan, along with the appearance of a new peak at 286.7 eV corresponding to C—S group. After covalent crosslinking with MoS₂ and PDA, most of the peak intensities of pullulan decreased along with a significant reduction in the peak intensity of C—S peak due to the formation C—S—Mo and C—S-PDA in the hydrogels. A similar observation was found in the deconvoluted Mo 3d spectra of the crosslinked hydrogels, where in addition to the characteristic peaks of Mo 3d at 232.73 eV (Mo 3d_{3/2}) and 229.63 eV (Mo 3d_{5/2}), there was an additional strong peak at 235.2 eV arising from the chemisorption of sulfur.

This was further supported by Raman analysis of crosslinked hydrogels (FIG. 2K). The Raman spectrum of Pul-SH showed presence of characteristic-SH peaks at 660 cm⁻¹ and 720 cm⁻¹ arising from C—S stretching vibrations, at 1120 cm⁻¹ from —SH bending and at 2610 cm⁻¹ corresponding to —SH stretching, respectively. Interaction of Pul-SH with PDA and MoS₂ showed significant reduction in the intensities of all these peaks in the single crosslinked and dual crosslinked hydrogels, which clearly establish the covalent crosslinking between thiol groups and sulfur-vacancies of MoS₂ on one hand, and with catechol groups of PDA on another.

### Elastomeric and Flexible Nanoengineered Hydrogel for 3D Printing

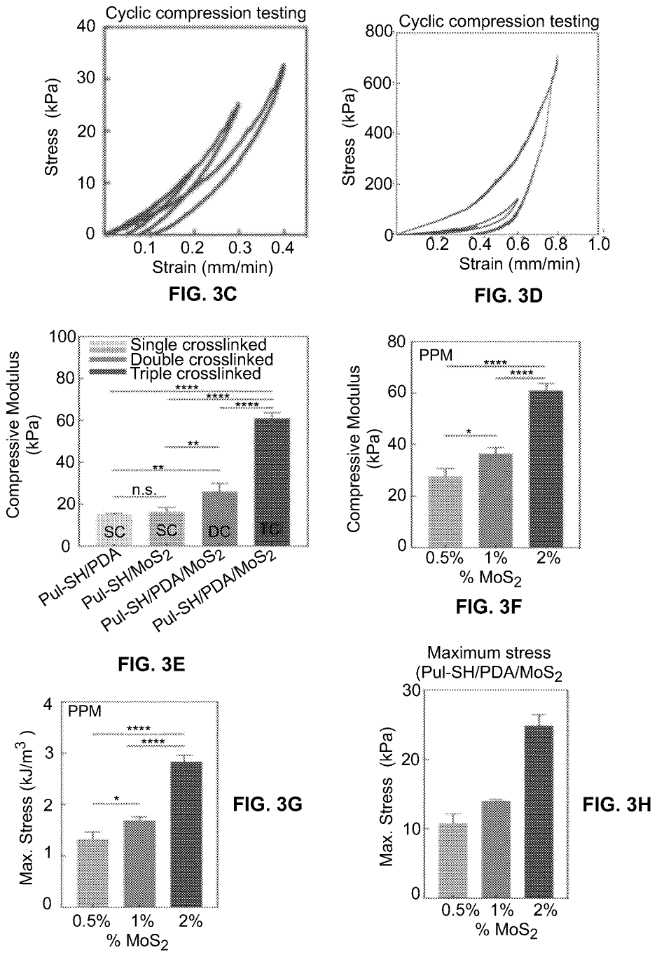

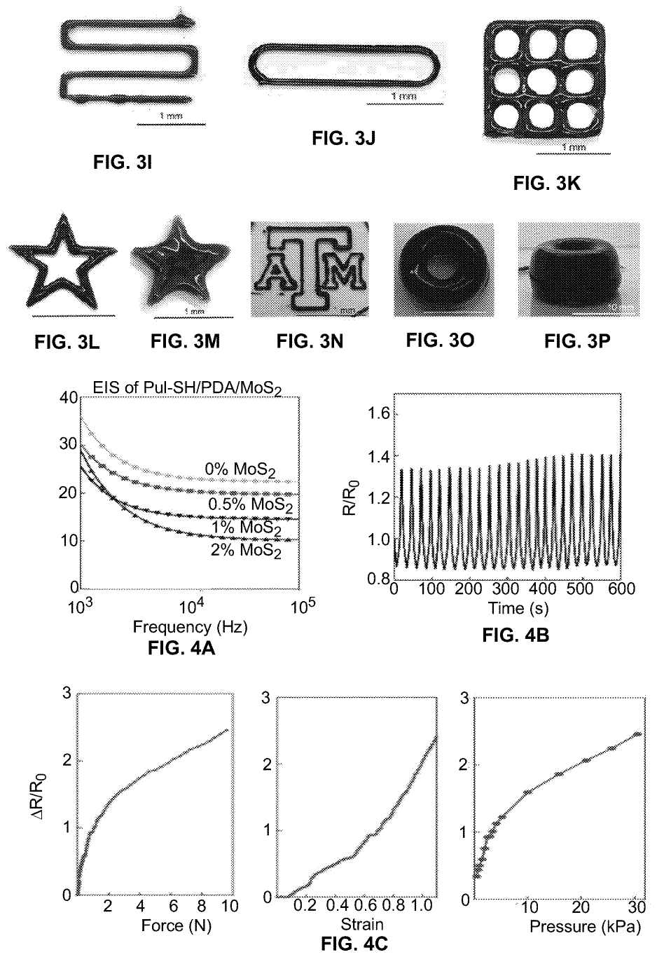

To be used as a flexible electronic skin, it is very important for the material to possess elastomeric properties to withstand any form of physical deformations such as dynamic stress and/or pressure, without damaging the crosslinked network. The mechanical resilience and robustness of the nanoengineered hydrogels were demonstrated through simple maneuvers including stretching, bending, compressing, and twisting (FIGS. 3A-3B). As shown in FIGS. 3A-3B, the hydrogel could withstand different deformations without fracturing and even be twisted 4 times without losing its physical integrity.

The tensile properties of the hydrogel were evaluated to understand its elastomeric characteristics. When subjected to tensile testing, the Pul-SH/2 wt % PDA/1 wt % MoS₂ hydrogel demonstrates impressive stretchability, indicating it can be easily extended under strain (FIGS. 3A-3B). The stress-strain curves showed that the nanocomposite hydrogel could be elongated upto 90% strain, demonstrating its excellent stretchability. The Young's modulus of the Pul-SH/2 wt % PDA/1 wt % MoS₂ hydrogel was calculated to be approximately 42 kPa. This value is notable as it falls within the range typical for physiological tissue, further highlighting its potential for bio-integration. However, it was also observed that decreasing the MoS₂ concentration to 0.5 wt % or increasing it to 2 wt % resulted in the hydrogel fracturing at 60% and 40% strain, respectively (FIGS. 3A-3B). This might be due to the formation of network defects in the hydrogel. Such defect could arise due to the absence of MoS₂ nanoassemblies for crosslinking with Pul-SH (0.5 wt %) or because of the presence of excess MoS₂ nanoassemblies (2 wt %) which contribute to formation of large network voids or defect for crack propogation. This showed that a concentration of 1 wt % of MoS₂ nanoassemblies was optimum in designing a mechanically flexible and elastomeric device.

Following this, uniaxial compression testing was conducted on a cylinder of Pul-SH/2 wt % PDA/1 wt % MoS₂ hydrogel with a height of 5 mm. (FIGS. 3C-3D). The hydrogel could withstand 80% compression and fully recover to its original shape and height without losing its physical integrity. The mechanical flexibility of the hydrogels was further investigated by subjecting the same hydrogel to five consecutive cycles of compressive testing by varying the strain from 20% to 80%. As can be seen from the FIGS. 3C-3D, the hydrogel showed complete recovery in each cycle, thereby demonstrating the mechanical robustness and elasticity of Pul-SH/PDA/MoS₂ hydrogels. From the stress-strain curves, the compressive modulus, toughness (FIGS. 3E-3G) and maximum stress (FIG. 3H) values of different hydrogel systems were calculated. Single crosslinking with either MoS₂ or PDA imparted a compressive modulus of ~16 kPa to the hydrogels, which improved to ~30 kPa with dual crosslinking and finally reached a compressive modulus of ~60 kPa in presence of triple crosslinking (FIGS. 3E-3G). Similarly, increasing concentrations of MoS₂ nanoassemblies from 0.5 wt % to 2 wt % also resulted in an increase in compressive strength of the crosslinked networks from 30 kPa to 60 kPa, respectively (FIGS. 3E-3G). Toughness (FIGS. 3E-3G) and maximum stress (FIG. 3H) values of the hydrogels also followed a similar trend of with increasing MoS₂ concentration. These results demonstrate that the hydrogel system exhibits elastomeric properties and robust inherent flexibility while demonstrating a stress profile in the kPa range which makes our e-skin more compliant for applications with soft body tissues (29, 30).

Interestingly, these nanoengineered pre-cursor solution (Pul-SH/2 wt % PDA/1 wt % MoS₂) are injected via 27G needles and can be stabilized by in situ gelation via covalent crosslinks, which allows the 3D printing of elastomeric devices. The printability of this nanoengineered pre-cursor was established by hanging filament method which indicated the ink can be extruded and recover post-extrusion withstanding 80% uniaxial compression. 3D printing of various basic shapes (FIGS. 31-3P) with accurate fidelity also was demonstrated such as rectangular curved lines, circular band, cross hatch pattern and hollow and filled star pattern. Finally, the Texas A&M University logo also was printe; ‘ATM’ and a hollow cylinder of height 7.5 mm, demonstrating post extrusion layer stability and bonding. These experiments demonstrate the printability of the ink and its suitability for designing 3D printed flexible structures.

### Dynamic Strain and Pressure Sensing Using 3D Printed Flexible Electronic Skin

There have been few reported works utilizing MoS₂ for designing flexible sensors (31-33). However, it is very important to highlight here that majority of these reports are not bioelectronic sensors for mimicking human skin, both in terms of flexibility, mechanical property and sensitivity. The reported works have utilized either monolayer MoS₂ deposited on Au electrode as a sensor, or polycrystalline MoS₂ thin film as the sensor, or as a coating material for polyurethane sponges to impart conductivity. None of the works utilized MoS₂ to design hydrogel-based flexible sensors with tunable mechanical properties that closely resemble human skin. Further, the reported studies utilize these platforms for either sensing strain or pressure. But none of the existing reports have demonstrated all strain, pressure and temperature responses in a single integrated flexible platform, which are the three crucial functions of human skin.

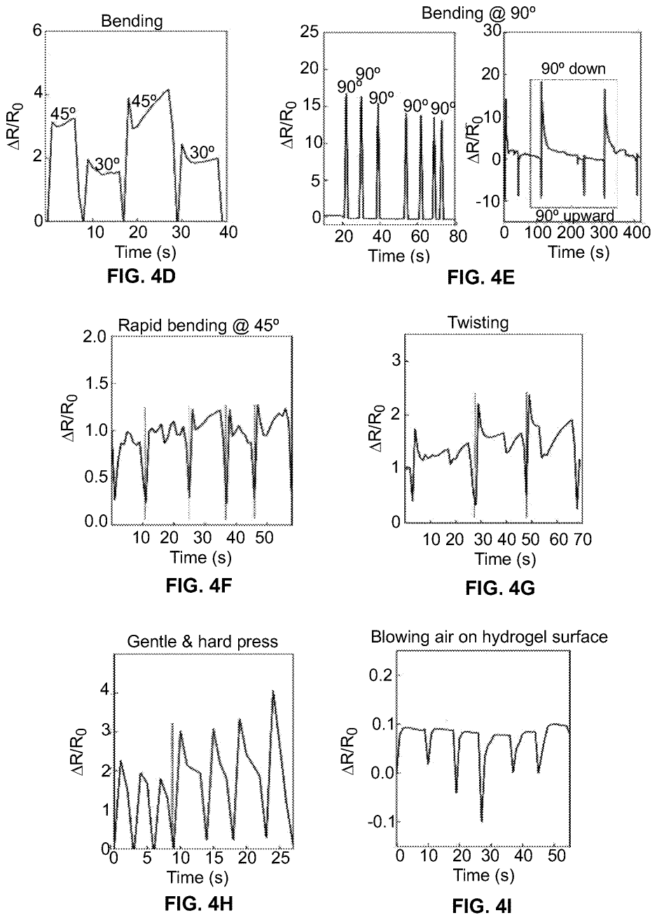

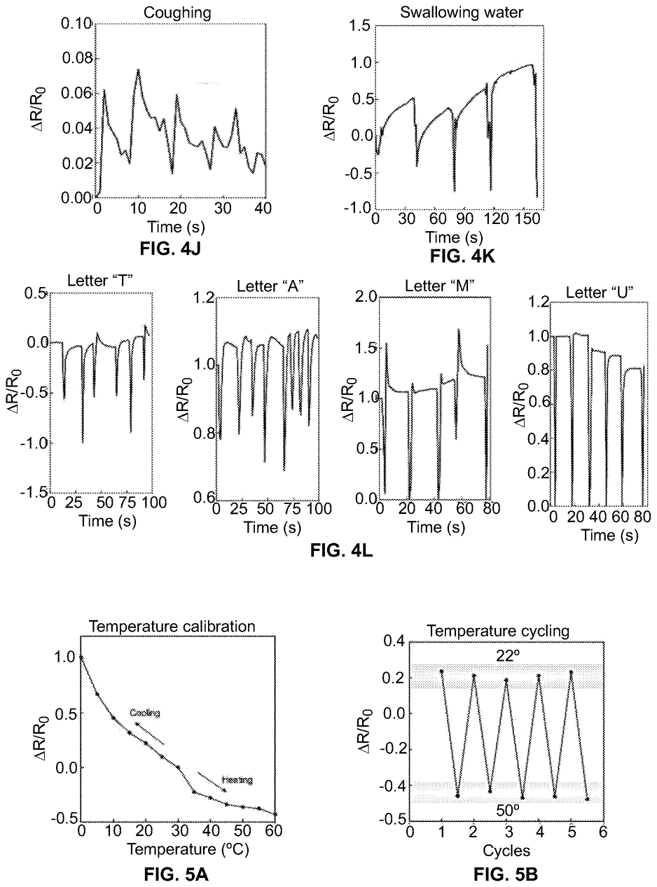

The 3D printed nanoengineered hydrogel (Pul-SH/2 wt % PDA/MoS₂) demonstrate significant electrical conductivity due to the presence of MoS₂ nanoassemblies. Impedance measurements through Electrical impedance spectroscopy (EIS) reported significant reduction in magnitude of impedance till 1 wt % MoS₂ indicating decrease in electrical resistance and thus, increase in conductivity (FIG. 4A). A subsequent increase in impedance at 2 wt % MoS₂ indicates reaching saturation limit with respect to MoS₂ concentration and no potential change in electrochemical properties of hydrogels. The hydrogel precursors were 3D printed and connected to a DC power supply to make a closed electrical circuit, making the LED bulb glow. The crosslinked hydrogels were also subsequently connected to the same circuit and were also able to successfully glow the LED bulbs (FIG. 4A). This demonstrates potential of utilizing these nanoengineered hydrogels for development of 3D printed electronic skin, actuators and electrically responsive delivery devices.

The exceptional elastomeric properties, electrical conductivity, and 3D-printability of the nanoengineered hydrogel spurred us to develop a 3D-printed electronic skin. This e-skin is designed to precisely monitor and sense dynamic changes in strain. As shown in FIGS. 4B-4C, the hydrogel (Pul-SH/2 wt % PDA/1 wt % MoS₂) was subjected to cyclic compression and its corresponding change in resistance values were recorded for each cycle over a period of time. The sensor responded to each loading and unloading steps of the compression cycle with a corresponding change in resistance, which showed very little deviation over time (FIGS. 4B-4C). This demonstrates that the electronic skin responded to the changes in strain accurately and with high sensitivity in a reproducible manner over multiple cycles of deformations. The sensitivity of a sensor relies heavily on the linearity of its response to changing external factors such as force, strain or pressure. The hydrogel responded to each change in force/strain/pressure and reflected a linear relationship between these factors and change in resistance (FIGS. 4B-4C).

For real-time application as an electronic skin, a wearable device was fabricated by encasing 3D printed hydrogels between two thin layers of PDMS and was used to monitor a wide range of human motions such as bending and twisting. The changes in resistance were measured based on the bending of the wearable device at 30°, 45° and 90° and twisting motions with repetitive cycles. The bending analysis revealed that the degree of deformation largely influenced the change in resistance. As shown in FIGS. 4D-4G, the bending of the wearable device at 30° as well as 45° could be differentiated based on the magnitude of the resistance change. For example, bending at 30° resembled less change in resistance in comparison to bending at 45°. Whereas, double bending at 90° gave rise to more prominent and sharp signals, and even greater change in resistance (FIGS. 4D-4G), showing precise sensitivity towards different degrees of deformity. Also, multiple 90° double bends in upward and downward direction during a continuous bending cycle yielded characteristic resistance changes in the hydrogel which was reproduced over consecutive cycles of bending. Further, the rapid single-side bending of the wearable device at 45° could also be sensed precisely by the device (FIGS. 4D-4G). Furthermore, the repeated twisting movement of the hydrogel also gave rise to specific resistance change signatures as shown in (FIGS. 4D-4G). Thus, these results clearly demonstrate the potential of using the nanoengineered pullulan hydrogel as an electronic skin for accurately sensing different human motions and patterns.

The 3D printed nanoengineered hydrogels can also respond to dynamic pressure through the change in resistance, indicating its potential for usage as dynamic pressure sensors. The hydrogel sensor was able to accurately respond to gentle and hard finger press (FIGS. 4H-4K) demonstrating its sensitivity to variable magnitudes of pressure. Remarkably, the hydrogels could detect blowing of air on its surface through a change in its resistance ((FIGS. 4H-4K). The nanoengineered hydrogels sensors were also able to recognize subtle movements including vocal cord vibrations and demonstrate the potential to be utilized as wearable phonatory-recognition device for vocal recovery and phonatory rehabilitation. The sensors were attached to throat, and it was able to accurately sense various vocal cord movements involved in coughing and drinking/swallowing water ((FIGS. 4H-4K). Finally, the elastomeric hydrogel sensor also demonstrates application as flexible touch keyboard and is able to identify writing action of individual letters written on the hydrogel device. Individual letters of the word ‘TAMU’ were written in a repeated action and the sensor was able to detect the letters through a characteristic resistance change (FIG. 4L). Overall, these applications demonstrate great potential of the 3D printed hydrogel sensors in detecting dynamic pressure and opens up its utilization as an electronic skin capable of sensing and responding to senses of touch, pressure and phonatory vibrations.

### 3D Printed Electronic Skin as a Flexible Temperature Sensor

In addition to strain and pressure sensing, the developed 3D printed electronic skin was also explored as a wearable temperature sensor for real-time monitoring and sensing of body temperature. In order to ensure heat preservation for accurate temperature measurements, fluctuations due to heat dissipation to surroundings was minimized by encasing the 3D printed hydrogel system between thin PDMS layers, which acted as thermal isolation material. The nanoengineered pullulan hydrogel demonstrated a highly linear temperature responsiveness over a broad range of temperature.

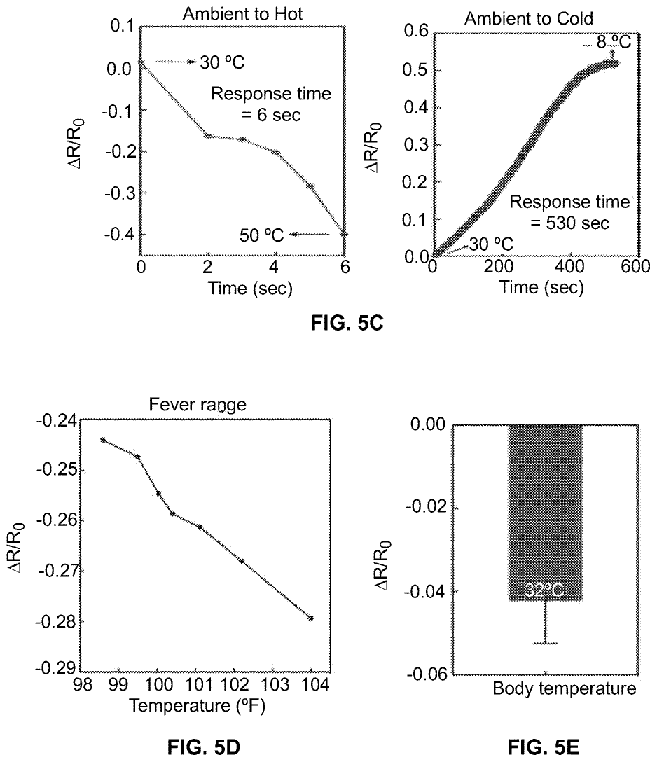

As shown in FIG. 5A, during both low (cooling) and high (heating) temperature ranges, a linear negative trend is observed in terms of relative resistance change in the hydrogel. Moreover, the hydrogel could also differentiate between hot (50° C.) and cold (22° C.) temperatures over repeated cycles and responded to the change in temperature with a corresponding resistance change (FIG. 5B), which showed negligeable deviation over the number of cycles tested. This showed the high sensitivity, stability and reproducibility of the hydrogel sensor in sensing differences in temperature. Further, the transient thermosensitive ability of the hydrogel was monitored for two different temperature ranges, namely ambient to hot and ambient to cold (FIG. 5C). It was observed that upon approaching a hot source (50° C.) from ambient (30° C.) temperature, the relative resistance change values of the hydrogel decreased very quickly (6 s) due to the negative temperature coefficient of the hydrogel. However, when the hydrogel approached a cold source (8° C.), there was a slow (550 s) and steady increase in the sensor resistance.

As a proof-of-concept demonstration, the nanoengineered electronic skin was used for real-time monitoring of human body temperature. As shown in FIG. 5D, the hydrogel could accurately sense the human fever range from 98° F.-104° F., where every 0.5° F. change in temperature could be precisely mapped with a corresponding change in resistance of the sensor. Finally, the electronic skin was utilized to measure the body temperature of a person (FIG. 5E), which demonstrates the potential of utilizing the developed nanoengineered hydrogel as flexible thermometer. The advantage of the designed e-skin as a temperature sensor lies in its property to behave as a “flexible” temperature sensor in contrast to a “rigid, non-flexible, non-conformable” traditional sensor. The flexibility which this e-skin imparts to this hydrogel-based temperature sensor makes it much more superior in terms of its utilization as a temperature sensor that can be conformally attached to any curved surfaces or convoluted topographies relevant to human body for real-time monitoring of temperature variation of a patient/individual in practical applications. 3D printing helps us to design such flexible temperature sensors in any design, shape and morphology as per the requirement, thereby making it more relevant for real-time applications.

The following references are cited herein.
1. Gao et al. Nature 2016, 529 (7587): 509-514.
2. Oh et al. Nature 2016, 539 (7629): 411-415.
3. Huang et al. Advanced Functional Materials 2019, 29 (6): 1805924.
4. Huh et al. Advanced Materials 2020, 32 (51): 2002092.
5. Wen et al. Materials Today 2018, 21 (2): 164-177.
6. Liu et al. Nature communications 201: 6 (1): 1-9.
7. Wang et al. Nature 2018, 555 (7694): 83-88.
8. Gao, L., Small 2017, 13 (35): 1603994.
9. Sun et al. Journal of Materials Chemistry A 2020, 8 (36): 18605-18623.
10. Deo et al. Acs Nano 2022, 16 (6): 8798-8811.
11. Karolina Pierchala et al. Acs Nano 2021, 15 (6): 9531-9549.
12. Wei et al. Chemistry of Materials 2021, 33 (17): 6731-6742.
13. Wang et al. Chemical Society Reviews 2023.
14. Hu et al. Advanced Materials 2022, 2205326
15. Shin et al. Small 2016, 12 (27): 3677-3689.
16. Zhang et al. Advanced Materials 2020, 32 (1): 1904752.
17. Vashist et al. Advanced healthcare materials 2018, 7 (9): 1701213.
18. Yang et al. Advanced Functional Materials 2017, 27 (40): 1703174.
19. Thoniyot et al. Advanced Science 2015, 2 (1-2): 1400010.
20. Shi et al. Advanced Materials 2017, 29 (22): 1603922.
21. Xu et al. Advanced Healthcare Materials 2021, 10 (11): 2100012.
22. Jaiswal et al. Advanced Materials 2017, 29 (36): 1702037.
23. Lee et al. Advanced Materials 2021, 33 (23): 2101238.
24. Lee et al. Science 2007, 318 (5849): 426-430.
25. Saeaeh et al. European Polymer Journal 2019, 120:109231.
26. Rodell et al. Advanced functional materials 2015, 25 (4): 636-644.
27. Soni et al. ACS Omega 2018, 3 (9): 11993-12009.
28. Deo et al. ACS Nano 2022
29. Rivnay et al. Science Advances 2017, 3 (6): e1601649.
30. Lacour et al. Nature Reviews Materials 2016, 1 (10): 1-14.
31. Chen et al. ACS Applied Materials & Interfaces 2023, 15 (1): 2043-2053.

What is claimed is:

1. A hydrogel, comprising:
a triple crosslinked network of:
a fungal exopolysaccharide modified with thiol groups;
a plurality of 2D transition metal dichalcogenides nanoassemblies having a plurality of sulfur vacancies; and
a plurality of nanoparticles structured with adhesive properties incorporated into the crosslinked fungal exopolysaccharide-transition metal dichalcogenides.

2. The hydrogel of claim 1, wherein the modified fungal exopolysaccharide is a thiolated pullulan (Pul-SH).

3. The hydrogel of claim 1, wherein the 2D transition metal dichalcogenides nanoassemblies comprise molybdenum disulfide (MoS₂).

4. The hydrogel of claim 1, wherein the plurality of nanoparticles are polydopamine (PDA) nanoparticles.

5. The hydrogel of claim 1, comprising a crosslinked pullulan-SH/polydopamine/molybdenum disulfide network (Pul-SH/PDA/MoS₂).

6. A method for nanoengineering a triple crosslinked hydrogel network for 3D-printing of an electronic skin, comprising:
modifying pullulan with thiols to form a thiolated pullulan;
incorporating sulfur vacancies in molybdenum disulfide nanoassemblies to form a defect-rich molybdenum disulfide;
crosslinking the thiolated pullulan with the defect-rich molybdenum disulfide to form a Pul-SH/MoS₂ network;
crosslinking polydopamine with the thiolated pullulan in the Pul-SH/MoS₂ network to form a Pul-SH/PDA/MoS₂ network; and
crosslinking pullulan chains in the Pul-SH/PDA/MoS₂ network via sodium trimetaphosphate, thereby nanoengineering the triple-crosslinked hydrogel network.

7. The method of claim 6, further comprising printing the triple crosslinked hydrogel network to produce a 3D-printed electronic skin.

8. A 3D-printed electronic skin produced by the method of claim 7.

9. The 3D-printed electronic skin of claim 8, comprising an integrated dynamic strain, pressure, and temperature sensor.

10. The 3D-printed electronic skin of claim 9 that is a flexible strain sensor configured to detect different dynamic human motions.

11. The 3D-printed electronic skin of claim 9 that is a wearable phonatory recognition platform and flexible touch keypad configured to detect precise changes in pressure.

12. The 3D-printed electronic skin of claim 9 that is a flexible thermometer configured to detect precise changes in temperature.

13. The 3D-printed electronic skin of claim 8, wherein the 3D-printed electronic skin has the properties of flexibility, stretchability, adhesion, moldability, and electrical conductivity when adhered to human skin.

14. A wearable electronic skin comprising a hydrogel network of thiolated pullulan triple crosslinked via reactions with defect-rich molybdenum disulfide, polydopamine and sodium trimetaphosphate, said crosslinked hydrogel network having a structure of a Pul-SH/PDA/MoS₂ network, and at least one electronic biosensor integrated therein.

15. The wearable electronic skin of claim 14, wherein the electronic biosensor is configured as a motion tracker, thermometer, phonatory-recognition platform, or touchpad or a combination thereof.

16. The wearable electronic skin of claim 14, wherein the Pul-SH/PDA/MoS₂ network is conformable to human skin.

17. A method for detecting and monitoring physiological changes through skin of a subject, comprising:
adhering the wearable electronic skin of claim 14 to the skin of the subject;
detecting at least one physiological change in the subject via the at least one electronic biosensor; and
monitoring the at least one physiological change over a period of time.

18. The method of claim 17, further comprising recording the physiological change.

19. The method of claim 17, further comprising diagnosing a disease or disorder based on the at least one physiological change in the subject.

20. The method of claim 17, wherein the at least one electronic biosensor detects human motions, senses touch, phonatory vibrations and body temperature.
